(19) United States
(12) Patent Application Publication
(10) Pub. No.: US 2017/0224215 A1
(43) Pub. Date: Aug. 10, 2017

(54) WARD CLOUD SYSTEM AND METHOD FOR CALIBRATING TIME AND ACCURACY OF INTELLIGENT ELECTRONIC VITAL-SIGN MONITORING DEVICE THEREOF

(71) Applicant: OSTAR MEDITECH CORP., New Taipei City (TW)

(72) Inventor: Kuo-Yuan CHANG, New Taipei City (TW)

(21) Appl. No.: 15/494,093

(22) Filed: Apr. 21, 2017

Related U.S. Application Data
(63) Continuation-in-part of application No. 14/159,439, filed on Jan. 21, 2014.

(30) Foreign Application Priority Data
Jan. 18, 2013 (TW) 102102038

(51) Int. Cl. A61B 5/00; G06F 19/00; A61B 5/145; A61B 5/0205; A61B 5/0402

(52) U.S. Cl. CPC A61B 5/0022; A61B 5/0205; A61B 5/7203; A61B 5/742; A61B 5/0402; A61B 5/14542; A61B 5/7475; G06F 19/322; A61B 5/021

(57) ABSTRACT

A ward cloud system uses an intelligent cloud database to receive and integrate vital-sign information detected by an intelligent electronic vital-sign monitoring device. The ward cloud system can receive time information and calibration curves from the far-end intelligent cloud database to synchronize the intelligent electronic vital-sign monitoring device and check and calibrate accuracy of the intelligent electronic vital-sign monitoring device. A method for calibrating time and accuracy of an intelligent electronic vital-sign monitoring device is disclosed also. The user needn't send the intelligent electronic vital-sign monitoring device back to the manufacturer for calibration with special equipment or extra manual setting. Therefore, labor and money is saved. The intelligent cloud database can analyze and integrate vital-sign information and file the vital-sign information into historical records. Therefore, testees, family members, physicians and nursing personnel can monitor the vital-sign information of patients in real time.

### CROSS REFERENCE TO RELATED APPLICATIONS

This application is a Continuation-in-Part of co-pending application Ser. No. 14/159,439, filed on Jan. 21, 2014, for which priority is claimed under 35 U.S.C. §120; and this application claims priority of Application No. 102102038 filed in Taiwan on Jan. 18, 2013 under 35 U.S.C. §119; the entire contents of all of which are hereby incorporated by reference.

### BACKGROUND OF THE INVENTION

Field of the Invention

The present invention relates to a ward cloud system, particularly to a ward cloud system calibrating time and accuracy automatically and using an intelligent cloud database to analyze and integrate vital-sign values.

Description of the Related Art

Most of the commercially-available physiological measurement devices or physiological monitors can measure, calculate and display. However, they lack functions of cloud operation, clock synchronization, and self-calibration. Therefore, they need self-calibrating and clock-synchronizing after they have been used for a period of time.

For example, the physicians administrate medication according to the results of blood pressure measurements, and the inpatients take blood pressure medicine on time. Errors of the instrument clocks may cause the physicians to make wrong medicine administration or cause the patients to take medicine in wrong timing. Clock non-synchronicity between instruments and medical personnel may cause confusion of treatment and nursing or even endanger the lives of patients. For example, the time of physician prescription is earlier than the time of blood pressure measurement, and the patients take antihypertensive medicine while they are in a low-blood pressure state.

Some regions have great temperature difference. For example, in the northeast of China, the temperature is −27° C. outdoors and 18° C. indoors. The circuit board of an electronic hemadynamometer is distorted outdoors and distorted once again after it has been taken back to the house and restored to the indoor temperature. Thus, the electronic hemadynamometer may have measurement errors. The circuit board of an electronic hemadynamometer may also be distorted after it has been stored for a long time. Then, the electronic hemadynamometer may also have measurement errors. In a high altitude, the calibration values of an electronic hemadynamometer may become inaccurate. Hence, an electronic hemadynamometer cannot undertake a precise measurement in a high altitude area. In the abovementioned cases, the electronic hemadynamometers need sending to the manufacturer for recalibration, which is money-, time- and labor-consuming and besets the users who need to monitor their blood pressure every day.

### SUMMARY OF THE INVENTION

In order to solve the abovementioned problems, the present invention proposes a ward cloud system and a method for calibrating time and accuracy of an intelligent electronic vital-sign monitoring device thereof, which use a far-end clock synchronization function to automatically synchronize the time of the intelligent electronic vital-sign monitoring device and the time of the intelligent cloud database, and which can also check and calibrate the accuracy of the intelligent electronic vital-sign monitoring device from a far end, whereby the user is exempted from sending the intelligent electronic vital-sign monitoring device back to the manufacturer for calibration with special equipment or additional manual programming, wherefore consumption of labor and money is reduced.

One objective of the present invention is to provide a ward cloud system, which transmits first vital-sign information to an intelligent cloud database for analyzing and integrating the first vital-sign information and establishing historical records of the first vital-sign information, wherein the testees, family members, physicians and nursing personnel can view and retrieve the vital-sign information through the network after they log in a far-end login interface, whereby the vital-sign information of patients can be monitored in real time.

To achieve the abovementioned objectives, a ward cloud system according to one embodiment of the present invention includes at least one intelligent electronic vital-sign monitoring device and an intelligent cloud database. The intelligent electronic vital-sign monitoring device includes a detecting module, an ID scanner, a first save module, a transmission module and a first processor, wherein the first processor is electrically connected with the detecting module, the ID scanner, the first save module and the transmission module, and wherein a machine serial number and a clock synchronization instruction of the intelligent electronic vital-sign monitoring device are saved in the first save module beforehand. The intelligent cloud database includes a second processor and a second save module, wherein the second save module is electrically connected with the second processor and saves a plurality of machine serial numbers of the intelligent electronic vital-sign monitoring devices and a plurality of mathematical functions of original zero point correction curves that are provided by manufacturers and corresponding to the machine serial numbers. The ward cloud system undertakes a measurement method that comprises a calibration process and a detection process.

Wherein the calibration process includes steps: while the intelligent electronic vital-sign monitoring device is started, the detecting module detects a no-load value functioning as a zero point data. The transmission module tries to establish a connection with the intelligent cloud database. If the connection is established, the intelligent electronic vital-sign monitoring device links with the intelligent cloud database. The first processor encodes the machine serial number and the clock synchronization instruction and then transmits the machine serial number and the clock synchronization instruction to the intelligent cloud database through the transmission module. The second processor decodes the machine serial number and the clock synchronization instruction. The second processor encodes a cloud time and a mathematical function of original zero point correction curve corresponding to the machine serial number, and then transmits the cloud time and the mathematical function of original zero point correction curve to the intelligent electronic vital-sign monitoring device. The first processor decodes the cloud time and the mathematical function of original zero point correction curve. The first processor compares the cloud time with the original time of the intelligent electronic vital-sign monitoring device to set the time of the intelligent electronic vital-sign monitoring device. The first processor compares the mathematical function of original zero point correction curve with the zero point data and calibrates the accuracy of the intelligent electronic vital-sign monitoring device;

Wherein the detection process includes steps: the ID scanner records ID information of a patient. The detecting module detects first vital-sign information of the patient, wherein the vital-sign information includes heartbeat waveform information. The first processor encodes the ID information and the first vital-sign information and then uploads the ID information and the first vital-sign information to the intelligent cloud database through the transmission module. The second processor decodes the ID information and the first vital-sign information and uses a Fourier analysis method to analyze whether there is noise in the heartbeat waveform information.

A method for calibrating time and accuracy of an intelligent electronic vital-sign monitoring device according to one embodiment of the present invention, comprises steps: providing at least one intelligent electronic vital-sign monitoring device including a detecting module, a first save module, a transmission module and a first processor, wherein the first processor is electrically connected with the detecting module, the first save module and the transmission module, and wherein a machine serial number and a clock synchronization instruction of the intelligent electronic vital-sign monitoring device are saved in the first save module beforehand. Providing an intelligent cloud database including a second processor and a second save module, wherein the second save module is electrically connected with the second processor and saves a plurality of machine serial numbers of the intelligent electronic vital-sign monitoring devices and a plurality of mathematical functions of original zero point correction curves that are corresponding to the machine serial numbers.

While the intelligent electronic vital-sign monitoring device is started, the detecting module detects a no-load value functioning as a zero point data, and the intelligent electronic vital-sign monitoring device links with the intelligent cloud database through the transmission module. The first processor encodes the machine serial number and the clock synchronization instruction and then uploads the machine serial number and the clock synchronization instruction to the intelligent cloud database through the transmission module. The second processor decodes the machine serial number and the clock synchronization instruction; the second processor encodes a cloud time and a mathematical function of original zero point correction curve corresponding to the machine serial number, and then transmits the cloud time and the mathematical function of original zero point correction curve to the intelligent electronic vital-sign monitoring device. The first processor decodes the cloud time and the mathematical function of original zero point correction curve. The first processor compares the cloud time with the original time of the intelligent electronic vital-sign monitoring device; if difference between the cloud time and the original time of the intelligent electronic vital-sign monitoring device is less than a given value, the intelligent electronic vital-sign monitoring device continues using the original time; if difference exceeds a given value, the time of the intelligent electronic vital-sign monitoring device is set to be the cloud time. The first processor compares the mathematical function of original zero point correction curve with the zero point data; if the zero point data is different from the benchmark of the mathematical function of original zero point correction curve, the mathematical function of original zero point correction curve takes the place of the zero point data to function as a measurement benchmark.

### BRIEF DESCRIPTION OF THE DRAWINGS

FIG. 1 is a block diagram schematically showing the structure of a ward cloud system according to one embodiment of the present invention;

FIG. 2 is a flowchart of a measurement method of a ward cloud system according to one embodiment of the present invention; and

FIG. 3 is a flowchart of a method for calibrating time and accuracy of an intelligent electronic vital-sign monitoring device according to one embodiment of the present invention.

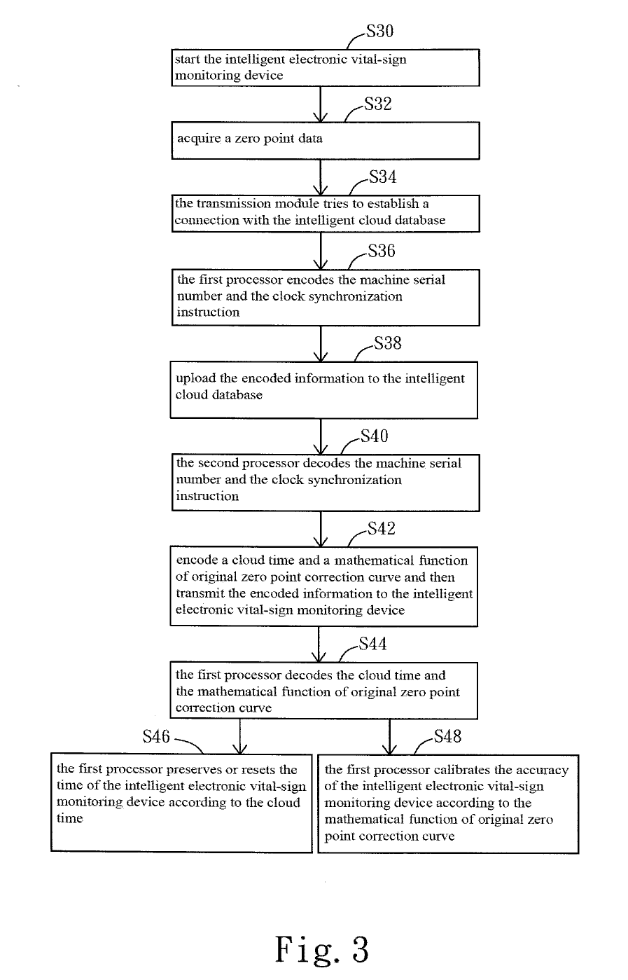

### DETAILED DESCRIPTION OF THE INVENTION

Below, the present invention is described in detail with embodiments. However, it should be understood that these embodiments are only to exemplify the present invention but not to limit the scope of the present invention.

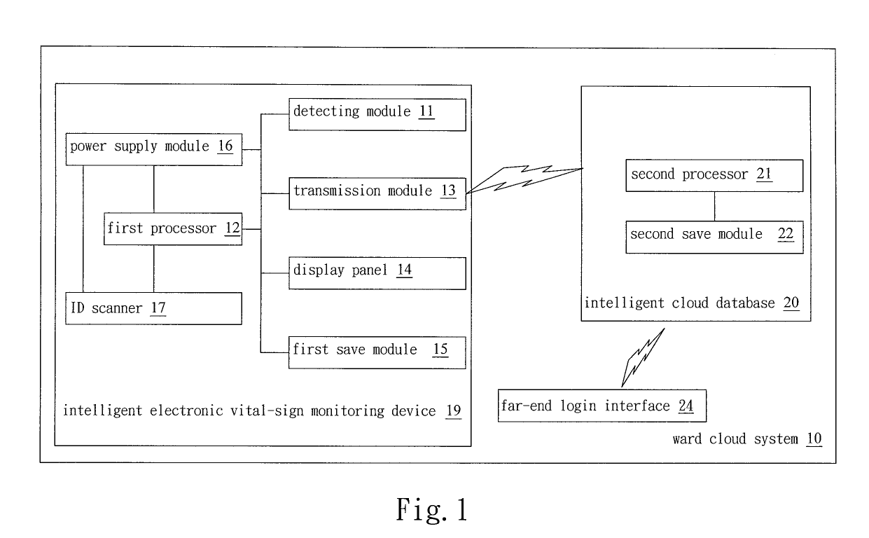

Refer to FIG. 1 a block diagram schematically showing the structure of a ward cloud system according to one embodiment of the present invention. The ward cloud system 10 comprises at least one intelligent electronic vital-sign monitoring device 19 and an intelligent cloud database 20. The intelligent electronic vital-sign monitoring device 19 includes a detecting module 11, an ID scanner 17, a first save module 15, a transmission module 13 and a first processor 12. The first processor 12 is electrically connected with the detecting module 11, the ID scanner 17, the first save module 15 and the transmission module 13. A machine serial number and a clock synchronization instruction of the intelligent electronic vital-sign monitoring device 19 are saved in the first save module beforehand. The intelligent cloud database 20 includes a second processor 21 and a second save module 22. The second save module 22 is electrically connected with the second processor 21, saving a plurality of machine serial numbers of the intelligent electronic vital-sign monitoring devices 19 and a plurality of mathematical functions of original zero point correction curves that are provided by the manufacturers and corresponding to the machine serial numbers.

A plurality of intelligent electronic vital-sign monitoring devices 19 respectively have their own machine serial numbers. Before each intelligent electronic vital-sign monitoring device 19 is delivered from the manufacturer, standard metrological instruments are used to calibrate a no-load value and more than one other values detected by the detecting module 11 of the intelligent electronic vital-sign monitoring device 19, whereby to establish a mathematical function of original zero point correction curve. In one embodiment, the first processor 12 uses a standard metrological instrument to convert the analog signals, which are detected in calibration, into digital signals of voltage to establish a mathematical function of original zero point correction curve. The mathematical functions of original zero point correction curves of the intelligent electronic vital-sign monitoring devices 19 of the same manufacturer are not necessarily the same, varying with the environments and the errors of electronic elements.

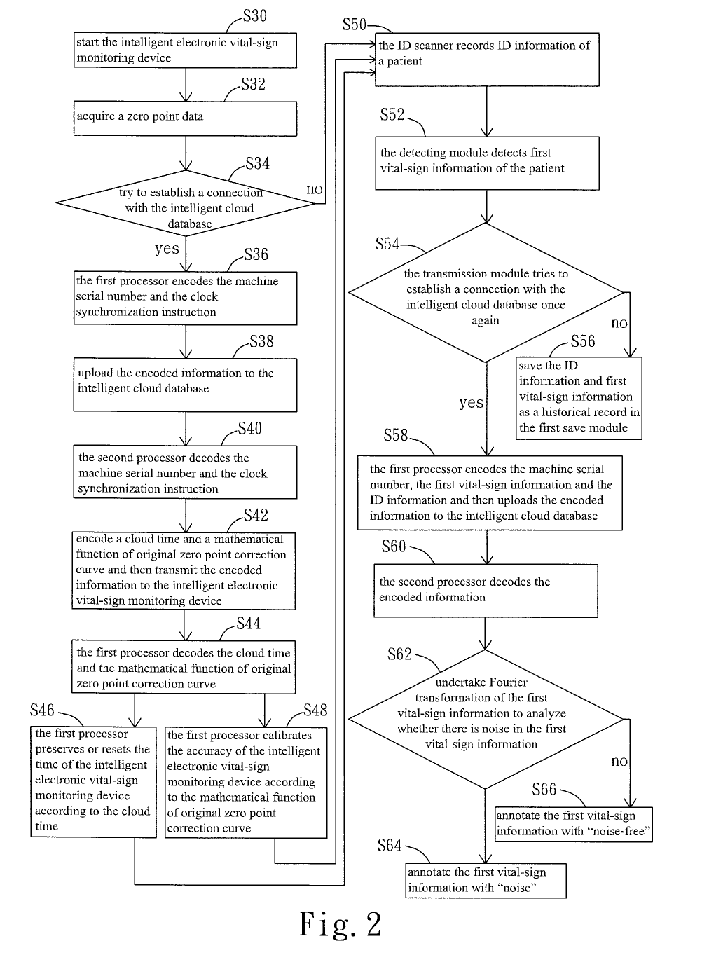

Refer to FIG. 2 a flowchart of a measurement method of a ward cloud system according to one embodiment of the present invention. The measurement method includes a calibration process and a detection process. The calibration process includes Steps S30-S48. In Step S30, start the intelligent electronic vital-sign monitoring device 19. In Step S32, the detecting module 11 detects a no-load analog signal, and the first processor 12 converts the analog signal into a value functioning as a zero point data. In one embodiment, the zero point data is a voltage value. In Step S34, the transmission module 13 tries to establish a connection with the intelligent cloud database 20. If the connection is established, the process proceeds to Step S36. If the transmission module 13 fails to establish the connection, undertake the detection process. In Step S36, the intelligent electronic vital-sign monitoring device 19 links with the intelligent cloud database 20, and the first processor 12 encodes the machine serial number and the clock synchronization instruction saved in the first save module 15. In one embodiment, the first processor 12 undertakes encoding in an advanced encryption standard (AES) method. In Step S38, the transmission module 13 uploads the encoded machine serial number and the encoded clock synchronization instruction to the intelligent cloud database 20. In Step S40, the second processor 21 decodes the machine serial number and the clock synchronization instruction. In one embodiment, the second processor 21 undertakes decoding in an AES method. In Step S42, according to the decoded machine serial number and clock synchronization instruction, the second processor 21 encodes a cloud time and a mathematical function of original zero point correction curve corresponding to the machine serial number and then transmits the encoded cloud time and the encoded mathematical function of original zero point correction curve to the intelligent electronic vital-sign monitoring device 19.

In Step S44, after the intelligent electronic vital-sign monitoring device 19 receives the encoded cloud time and the encoded mathematical function of original zero point correction curve, the first processor 12 decodes the cloud time and the mathematical function of original zero point correction curve. In Step S46, the first processor 12 compares the cloud time with the original time of the intelligent electronic vital-sign monitoring device 19 to determine whether the time of the intelligent electronic vital-sign monitoring device 19 is preserved or reset. In Step S48, the first processor 12 compares the mathematical function of original zero point correction curve with the zero point data and calibrates the accuracy of the intelligent electronic vital-sign monitoring device 19.

After the intelligent electronic vital-sign monitoring device 19 completes time calibration and accuracy calibration, the detection process is undertaken. The detection process includes Steps S50-S66. In Step S50, the ID scanner 17 records ID information of a patient. In Step S52, the detecting module 11 detects first vital-sign information of the patient. The vital-sign information includes heartbeat waveform information. As mentioned above, after the intelligent electronic vital-sign monitoring device 19 is started, if the transmission module 13 fails to establish a connection in Step S34, the process also proceeds to the detection process. In other words, if a connection is not established in Step S34, the system would not undertake time calibration and accuracy calibration of the intelligent electronic vital-sign monitoring device 19 but directly undertakes vital-sign detection after the user inputs the patient ID information. In Step S54, the transmission module 13 tries to establish a connection with the intelligent cloud database 20 once again. If the transmission module 13 fails to establish a connection, the process proceeds to Step S56. In Step S56, the ID information and first vital-sign information of the patient is saved as a historical record in the first save module 15. If the connection is established, the process proceeds to Step S58. In Step S58, the first processor 12 encodes the machine serial number, the first vital-sign information and the ID information and then uploads the machine serial number, the first vital-sign information and the ID information to the intelligent cloud database 20 through the transmission module 13. In Step S60, the second processor 21 decodes the first vital-sign information and the ID information. In Step S62, the second processor 21 undertakes Fourier transformation of the heartbeat waveform information to analyze whether there is noise in the heartbeat waveform information. If there is noise, the process proceeds to Step S64. In Step S64, the record of the patient is annotated with “heartbeat noise”. If there is no noise, the process proceeds to Step S66. In Step S66, the record of the patient is annotated with “heartbeat noise-free”.

In time calibration of Step S46, if the difference between the cloud time and the time of the intelligent electronic vital-sign monitoring device 19 is less than a given value, the intelligent electronic vital-sign monitoring device 19 continues using the original time. If the difference exceeds a given value, the intelligent electronic vital-sign monitoring device 19 turns to use the cloud time.

In accuracy calibration of Step S48, if the comparison shows that the zero point data is different from the mathematical function of original zero point correction curve, the mathematical function of original zero point correction curve takes the place of the zero point data to function as the measurement benchmark. If the comparison shows that the zero point data is identical to the mathematical function of original zero point correction curve, no accuracy calibration is undertaken.

In one embodiment, the ward cloud system 10 further comprises a far-end login interface 24, which allows the user to log in the intelligent cloud database 20 to analyze the heartbeat noise-annotated or heartbeat noise-free first vital-sign information, wherein the heartbeat waveform information of the first vital-sign information includes one or more of heart pulse information, blood pressure information, blood oxygen information, and electrocardiographic information. In one embodiment, the heartbeat waveform information of the first vital-sign information is a time-domain pulse diagram, which is converted into a frequency-domain energy spectrum by Fast Fourier Transform. The frequency-domain energy spectrum includes at least three frequency ranges: a first frequency range, a second frequency range and a third frequency range. The number of the non-zero spikes in each frequency range is calculated and defined as the noise value. The user can use the noise value to understand the heart status of the patient.

In one embodiment, the intelligent electronic vital-sign monitoring device 19 further comprises a display module 14, which is electrically connected with the first processor 12 and presents the first vital-sign information. In one embodiment, the display module 14 is a liquid crystal display (LCD) device or a light emitting diode (LED) display device. The transmission module 13 of the intelligent electronic vital-sign monitoring device 19 is a wired transmission interface (such as a universal serial bus (USB) port or a RS232 port) or a wireless transmission interface (such as a 2.4G WiFi module or a wireless local area network (LAN) module). The intelligent electronic vital-sign monitoring device 19 further comprises a power supply module 16. The power supply module 16 is connected with the detecting module 11, the ID scanner 17, the first save module 15, the transmission module 13, the display module 14 and the first processor 12, supplying power to charge batteries or to drive the system. In one embodiment, the power supply module 16 is an alkaline battery set, a rechargeable battery set, a capacitor or a power supply device.

In one embodiment, the intelligent cloud database 20 is a hospital information system (HIS), a nursing information system (NIS), a health level (HL) 7, or an ordinary intelligent cloud database system. The intelligent cloud database 20 can link with one or more of external systems, such as HIS systems, NIS systems, emergency department systems, intensive care unit systems and clinic systems, and exchange information with the abovementioned external systems.

The abovementioned operation process (including Steps S30-S48) of the ward cloud system 10 discloses a method for calibrating time and accuracy of an intelligent electronic vital-sign monitoring device. While the intelligent electronic vital-sign monitoring device 19 is started (Step S30), the detecting module 11 of the intelligent electronic vital-sign monitoring device 19 detects a no-load value functioning as a zero point data (Step S32). The transmission module 13 of the intelligent electronic vital-sign monitoring device 19 links with an intelligent cloud database 20 (Step S34). The first processor 12 of the intelligent electronic vital-sign monitoring device 19 encodes the machine serial number and the clock synchronization instruction (Step S36). The transmission module 13 uploads the encoded machine serial number and the encoded clock synchronization instruction to the intelligent cloud database 20 (Step S38). The second processor 21 of the intelligent cloud database 20 decodes the machine serial number and the clock synchronization instruction (Step S40). The second processor 21 encodes a cloud time and a mathematical function of original zero point correction curve corresponding to the machine serial number and transmits the encoded cloud time and the encoded mathematical function of original zero point correction curve to the intelligent electronic vital-sign monitoring device 19 (Step S42). The first processor 12 decodes the cloud time and the mathematical function of original zero point correction curve, which are to be used to calibrate time and accuracy (Step S44).

In calibrating time of the intelligent electronic vital-sign monitoring device 19, the first processor 12 compares the cloud time with the original time of the intelligent electronic vital-sign monitoring device 19 to determine whether the time of the intelligent electronic vital-sign monitoring device 19 is preserved or reset (Step S46). In one embodiment, if the difference between the cloud time and the time of the intelligent electronic vital-sign monitoring device 19 is less than a given value, the intelligent electronic vital-sign monitoring device 19 continues using the original time; If the difference exceeds a given value, the cloud time is set to be the time of the intelligent electronic vital-sign monitoring device 19.

In calibrating accuracy of the intelligent electronic vital-sign monitoring device 19, the first processor 12 compares the mathematical function of original zero point correction curve with the zero point data and calibrates the accuracy of the intelligent electronic vital-sign monitoring device 19 (Step S48). If the comparison shows that the zero point data is different from the mathematical function of original zero point correction curve, the mathematical function of original zero point correction curve takes the place of the zero point data to function as the measurement benchmark. If the comparison shows that the zero point data is identical to the mathematical function of original zero point correction curve, no accuracy calibration is undertaken. For example, suppose that the zero point value of the mathematical function of original zero point correction curve of the intelligent electronic vital-sign monitoring device 19 is 4.0V, and suppose that the detecting module 11 detects a no-load zero point value of 4.1V on starting the intelligent electronic vital-sign monitoring device 19; in such a case, if the zero point value of 4.0V is used in the mathematical function of original zero point correction curve, errors will occur; thus, the errors are offset to calibrate the accuracy of the device; thereby, the accuracy deviation of the intelligent electronic vital-sign monitoring device 19 itself would not affect the accuracy of the first vital-sign information detected by the detecting module 11 in the succeeding measurements.

In the present invention, the ward cloud system can receive a cloud time and a mathematical function of original zero point correction curve from a far end database, check time and accuracy of the intelligent electronic vital-sign monitoring device and then calibrate time and accuracy of the intelligent electronic vital-sign monitoring device, exempted from being sent back to the manufacturer for calibration with special equipment or additional manual setting, wherefore the user needn't spend extra money and labor in calibration. Further, the ward cloud system of the present invention transmits first vital-sign information to an intelligent cloud database. The intelligent cloud database analyzes and integrates the first vital-sign information and establishes historical records of the first vital-sign information. The testees, family members, physicians and nursing personnel can view and retrieve the vital-sign information through the network after they log in a far-end login interface. Thereby, the vital-sign information of patients can be monitored in real time.

Although the present invention has been explained in relation to its preferred embodiment, it is to be understood that other modifications and variation can be made without departing the spirit and scope of the invention as hereafter claimed.

What is claimed is:

1. A ward cloud system comprising
at least one intelligent electronic vital-sign monitoring device including a detecting module, an ID scanner, a first save module, a transmission module and a first processor, wherein the first processor is electrically connected with the detecting module, the ID scanner, the first save module and the transmission module, and wherein a machine serial number and a clock synchronization instruction of the intelligent electronic vital-sign monitoring device are saved in the first save module beforehand; and
an intelligent cloud database including a second processor and a second save module, wherein the second save module is electrically connected with the second processor and saves a plurality of machine serial numbers of the intelligent electronic vital-sign monitoring devices and a plurality of mathematical functions of original zero point correction curves that are provided by manufacturers and corresponding to the machine serial numbers;
wherein the ward cloud system undertakes a measurement method that comprises a calibration process and a detection process, and
wherein the calibration process includes steps:
while the intelligent electronic vital-sign monitoring device is started, the detecting module detects a no-load value functioning as a zero point data; the transmission module tries to establish a connection with the intelligent cloud database;
if the connection is established, the intelligent electronic vital-sign monitoring device links with the intelligent cloud database; the first processor encodes the machine serial number and the clock synchronization instruction and then transmits the machine serial number and the clock synchronization instruction to the intelligent cloud database through the transmission module;
the second processor decodes the machine serial number and the clock synchronization instruction; the second processor encodes a cloud time and a mathematical function of original zero point correction curve corresponding to the machine serial number, and then transmits the cloud time and the mathematical function of original zero point correction curve to the intelligent electronic vital-sign monitoring device; and
the first processor decodes the cloud time and the mathematical function of original zero point correction curve; the first processor compares the cloud time with the original time of the intelligent electronic vital-sign monitoring device to set the time of the intelligent electronic vital-sign monitoring device; the first processor compares the mathematical function of original zero point correction curve with the zero point data and calibrates the accuracy of the intelligent electronic vital-sign monitoring device;
wherein the detection process includes steps:
the ID scanner records ID information of a patient; the detecting module detects first vital-sign information of the patient, wherein the vital-sign information includes heartbeat waveform information;
the first processor encodes the ID information and the first vital-sign information and then uploads the ID information and the first vital-sign information to the intelligent cloud database through the transmission module; and
the second processor decodes the ID information and the first vital-sign information and uses a Fourier analysis method to analyze whether there is noise in the heartbeat waveform information.

2. The ward cloud system according to claim 1, wherein if there is noise in the heartbeat waveform information, the first vital-sign information annotated with heartbeat noise; if there is no noise in the heartbeat waveform information, the first vital-sign information annotated with heartbeat noise-free.

3. The ward cloud system according to claim 1, wherein while the first processor compares the mathematical function of original zero point correction curve with the zero point data, if the zero point data is different from the benchmark of the mathematical function of original zero point correction curve, the mathematical function of original zero point correction curve takes the place of the zero point data to function as a measurement benchmark.

4. The ward cloud system according to claim 1, wherein after the intelligent electronic vital-sign monitoring device is started, the transmission module tries to establish a connection with the intelligent database;
if the transmission module fails to establish a connection, the ward cloud system does not undertake the calibration process but directly undertakes the detection process: the ID scanner records the ID information of the patient, and the detecting module detects the first vital-sign information of the patient; and
after the detecting module detects the first vital-sign information, the transmission module tries to establish a connection with the intelligent cloud database once again; if the connection is established, the first processor encodes the ID information and the first vital-sign information and uploads the ID information and the first vital-sign information to the intelligent cloud database through the transmission module; the second processor decodes the ID information and the first vital-sign information and uses a Fourier analysis method to analyze whether there is noise in the heartbeat waveform information; if the transmission module still fails to establish a connection, the ID information and first vital-sign information of the patient is saved as a historical record in the first save module.

5. The ward cloud system according to claim 1, wherein after the first processor compares the cloud time with the original time of the intelligent electronic vital-sign monitoring device, if difference between the cloud time and the original time of the intelligent electronic vital-sign monitoring device is less than a given value, the intelligent electronic vital-sign monitoring device continues using the original time; if difference exceeds a given value, the time of the intelligent electronic vital-sign monitoring device is set to be the cloud time.

6. The ward cloud system according to claim 1, wherein before the intelligent electronic vital-sign monitoring device is delivered from the manufacturer, a standard metrological instrument is used to calibrate a no-load value and more than one other values detected by the detecting module to establish the mathematical function of original zero point correction curve.

7. The ward cloud system according to claim 1 further comprising a far-end login interface, which allows the user to log in the intelligent cloud database to analyze the heartbeat noise-annotated or heartbeat noise-free first vital-sign information.

8. The ward cloud system according to claim 1, wherein the intelligent electronic vital-sign monitoring device further includes a display module, which is electrically connected with the first processor and presents the first vital-sign information.

9. The ward cloud system according to claim 1, wherein the heartbeat waveform information includes one or more of heart pulse information, blood pressure information, blood oxygen information, and electrocardiographic information.

10. The ward cloud system according to claim 1, wherein the transmission module is a wireless transmission module.

11. The ward cloud system according to claim 1, wherein the intelligent cloud database includes a hospital information system (HIS), a nursing information system (NIS), a health level (HL) 7 database system, or an ordinary intelligent cloud database system.

12. The ward cloud system according to claim 1, wherein the intelligent cloud database can link with one or more of external systems, including HIS systems, NIS systems, emergency department systems, intensive care unit systems and clinic systems, and can exchange information with the external systems.

13. A method for calibrating time and accuracy of an intelligent electronic vital-sign monitoring device, comprising steps:
providing at least one intelligent electronic vital-sign monitoring device comprising a detecting module, a first save module, a transmission module and a first processor, wherein the first processor is electrically connected with the detecting module, the first save module and the transmission module, and wherein a machine serial number and a clock synchronization instruction of the intelligent electronic vital-sign monitoring device are saved in the first save module beforehand;
providing an intelligent cloud database including a second processor and a second save module, wherein the second save module is electrically connected with the second processor and saves a plurality of machine serial numbers of the intelligent electronic vital-sign monitoring devices and a plurality of mathematical functions of original zero point correction curves that are corresponding to the machine serial numbers; wherein
while the intelligent electronic vital-sign monitoring device is started, the detecting module detects a no-load value functioning as a zero point data, and the intelligent electronic vital-sign monitoring device links with the intelligent cloud database through the transmission module;
the first processor encodes the machine serial number and the clock synchronization instruction and then uploads the machine serial number and the clock synchronization instruction to the intelligent cloud database through the transmission module;
the second processor decodes the machine serial number and the clock synchronization instruction; the second processor encodes a cloud time and a mathematical function of original zero point correction curve corresponding to the machine serial number, and then transmits the cloud time and the mathematical function of original zero point correction curve to the intelligent electronic vital-sign monitoring device;
the first processor decodes the cloud time and the mathematical function of original zero point correction curve; the first processor compares the cloud time with the original time of the intelligent electronic vital-sign monitoring device; if difference between the cloud time and the original time of the intelligent electronic vital-sign monitoring device is less than a given value, the intelligent electronic vital-sign monitoring device continues using the original time; if difference exceeds a given value, the time of the intelligent electronic vital-sign monitoring device is set to be the cloud time; and
the first processor compares the mathematical function of original zero point correction curve with the zero point data; if the zero point data is different from the benchmark of the mathematical function of original zero point correction curve, the mathematical function of original zero point correction curve takes the place of the zero point data to function as a measurement benchmark.

14. The method for calibrating time and accuracy of an intelligent electronic vital-sign monitoring device according to claim 13, wherein before the intelligent electronic vital-sign monitoring device is delivered from the manufacturer, a standard metrological instrument is used to calibrate a no-load value and more than one other values detected by the detecting module to establish the mathematical function of original zero point correction curve.
